(19) United States
(12) Patent Application Publication
(10) Pub. No.: US 2020/0181611 A1
(43) Pub. Date: Jun. 11, 2020

(54) NUCLEIC ACIDS COMPRISING IMPREFECT HAIRPINS

(71) Applicant: Devgen NV, Gent-Zwijnaarde (BE)

(72) Inventors: Wendy MADDELEIN, Gent-Zwijnaarde (BE); Isabelle MAILLET, Gent-Zwijnaarde (BE)

(73) Assignee: Devgen NV, Gent- Zwijnaarde (BE)

(21) Appl. No.: 16/342,302

(22) PCT Filed: Oct. 19, 2016
(86) PCT No.: PCT/EP2016/075087

§ 371 Date: Apr. 16, 2019

(51) Int. Cl. C12N 15/11

(52) U.S. Cl. CPC C12N 15/111; C12N 2310/14; C12N 2310/531; C12N 2330/50; C12N 2320/53

(57) ABSTRACT

The present invention provides an RNA comprising in the 5′ to 3′ direction a first sequence and a second sequence, the first sequence being spaced apart from the second sequence by a spacer sequence, the first and second sequences being substantially complementary and forming a duplex via Watson and Crick base pairing, the second sequence being able to hybridise with the mRNA of a target gene in an organism, the RNA being substantially in the form of a hairpin, the second sequence being fully complementary to the said mRNA along the region at which it hybridises, the first sequence comprising nucleotide bases which do not Watson and Crick base pair with the corresponding bases in the second sequence of the duplex wherein the duplex does not comprise regions of 3 or more consecutive mismatched bases or an RNA comprising in the 5′ to 3′ direction a first sequence and a second sequence, the first sequence being spaced apart from the second sequence by a spacer sequence, the first and second sequences being substantially complementary and forming a duplex via Watson and Crick base pairing, the first sequence being able to hybridise with the mRNA of a target gene in an organism, the RNA being substantially in the form of a hairpin, the first sequence being fully complementary to the said mRNA along the region at which it hybridises, the second sequence comprising nucleotide bases which do not Watson and Crick base pair with the corresponding nucleotide bases in the first sequence of the duplex, wherein the duplex does not comprise regions of 3 or more consecutive mismatched bases.

The present invention relates to post-transcriptional gene silencing, particularly its use as a possible agent in the control of pest organisms, such as insect or other pests of cultivated crops.

The phenomenon of controlling gene expression by introducing into an organism or cell a double stranded RNA, one strand of which comprises a sequence which is complementary to an mRNA of a target essential-gene in the organism or cell is well known. The double stranded RNA may be produced by a number of known means. For example, where the dsRNA is produced by either an in vitro or in vivo transcription system, the DNA encoding it may comprise a single promoter which expresses both strands of the RNA duplex which are separated by a spacer which provides for the formation of a duplex. The sequences in the DNA are thus a first sequence and a second sequence which is the reverse complement of the first, separated by the spacer, and the dsRNA thus produced is in the form of a hairpin. Alternatively the first and second sequences of the dsRNA may be the transcription products of a double stranded DNA, each strand of which comprises a promoter which drives expression of the sequences in the corresponding 5 prime to 3 prime directions on the complementary sense and antisense strands of the DNA.

Whilst the provision of hairpin dsRNA from a DNA template is generally well known and practised, there are problems associated with such provision—in particular a reluctance of RNA polymerases to transcribe DNA which encodes a hairpin-dsRNA.

The present invention provides, at least to some extent, a solution to this problem.

According to the present invention there is provided an RNA comprising in the 5′ to 3′ direction a first sequence and a second sequence, the first sequence being spaced apart from the second sequence by a spacer sequence, the first and second sequences being substantially complementary and forming a duplex via Watson and Crick base pairing, the second sequence being able to hybridise with the mRNA of a target gene in an organism, the RNA being substantially in the form of a hairpin, the second sequence being fully complementary to the said mRNA along the region at which it hybridises, the first sequence comprising nucleotide bases which do not Watson and Crick base pair with the corresponding bases in the second sequence of the duplex wherein the duplex does not comprise regions of 3 or more consecutive mismatched bases.

The invention also provides a corresponding RNA comprising in the 5′ to 3′ direction a first sequence and a second sequence, the first sequence being spaced apart from the second sequence by a spacer sequence, the first and second sequences being substantially complementary and forming a duplex via Watson and Crick base pairing, the first sequence being able to hybridise with the mRNA of a target gene in an organism, the RNA being substantially in the form of a hairpin, the first sequence being fully complementary to the said mRNA along the region at which it hybridises, the second sequence comprising nucleotide bases which do not Watson and Crick base pair with the corresponding nucleotide bases in the first sequence of the duplex, wherein the duplex does not comprise regions of 3 or more consecutive mismatched bases.

For the avoidance of doubt, when length of a sequence is mentioned, what is meant is the number of consecutive nucleotides in the sequence which are joined together by phosphodiester or phosphorothionate bonds. Thus—the length of a duplex sequence may be 100 nucleotides—this means that there are 200 nucleotides in that sequence.

By being fully complementary to the said mRNA along the region at which it hybridises is meant that when either the first or second sequence (as the case may be) hybridises with the mRNA from a target gene, there are no positions within the hybridising region which contain any base mismatches—meaning that Adenine always base pairs with Uracil and Guanine always base pairs with Cytosine.

By duplex is meant double stranded.

The RNA according to the invention is in the form of a generally well-known stem/loop structure, also known as a hairpin.

By regions of 3 or more consecutive mismatched bases is meant 3 or more bases in the same sequence, the first base being immediately adjacent the second and joined to it by a phosphodiester (or phosphorothionate) bond, the second base being immediately adjacent to the third and likewise joined to it by a phosphodiester (or phosphorothionate) bond, all of the bases being unable to Watson and Crick pair with the corresponding bases in the otherwise complementary sequence of the duplex.

In a preferred embodiment of the RNA of the present invention, the mismatching bases are substantially equally spaced along the length of the duplex, at, for example, about every 5^{th} position, more preferably at about every 10^{th} position and still more preferably at about every 20^{th} position, although the present inventive RNA also embraces duplexes in which the mismatches are also randomly distributed along the length of the duplex, with the proviso mentioned above that the duplex does not comprise regions of 3 or more consecutive mismatched bases.

The skilled artisan will recognise the equally spaced intervals of mismatches described above do not necessarily start from the first nucleotide in any given duplex but may start from nucleotides further along the duplex.

The total number of base mismatches between the corresponding sequences in the duplex is not particularly limited, being dictated in large part by the length of the duplex. For example, where the duplex length is 100 nucleotides (the duplex thus comprising 200 nucleotides) about 30 percent of the nucleotides may not Watson and Crick base pair. Still more preferably about 20 percent, and still more preferably about 10 percent of the nucleotides in the duplex do not Watson and Crick base pair.

Whilst the bases in a sequence which do not Watson and Crick base pair with bases in the corresponding sequence of the duplex are A in the first sequence which corresponds with G, C or A in the second sequence, U in the first sequence which corresponds with G, C or U in the second sequence, C in the first sequence which corresponds with U, C or A in the second sequence, G in the first sequence which corresponds with U, G or A in the second sequence, the particularly preferred combinations are A which mismatches with C, U which mismatches with G, C which mismatches with A.

The RNA according to the present invention comprises a spacer sequence (otherwise known as a loop region in a hairpin structure), the length of which is sufficient to enable the first and second sequences to hybridise to form the duplex, or in other words stem region of a hairpin structure. Any length is possible, with the proviso that it should not be so long as to impede duplex formation. As is known in the art, the DNA region encoding this spacer region may comprise an intron. Preferably the spacer has a length of about 15 to about 350 nucleotides, more preferably a length of about 20 to about 100 nucleotides and most preferably a length of about 20 to about 50 nucleotides.

The length of the duplex in the hairpin structure may be at least 2,000 nucleotides, although shorter sequences are preferred, being typically up to 1500 nucleotides, or even shorter, being for example 100 to 500 nucleotides.

The length of the sequence which hybridises with the mRNA may be about 20 to about 100 nucleotides, more preferably about 20 to about 80 nucleotides, still more preferably about 20 to about 50 nucleotides, and most preferably about 20 to about 30 nucleotides.

Whilst the target gene may be any gene in a target organism, it is preferred that the gene is a so called “essential gene”, meaning that it is a gene, the correct expression of which is essential to the life or well-being of the organism. Most preferably, an essential gene is one, the suppressed expression of which leads to death of the organism. The target gene may be plant gene, the post-transcriptional silencing of which causes an improvement in an agronomic trait, such as yield or quality, or a viral gene, the silencing of which renders the virus substantially inactive. The skilled artisan is well aware of examples of these target genes. In a particularly preferred embodiment of the RNA according to the present invention, the target gene is an essential gene of a plant pest organism, such as an insect pest, for example any of the corn-rootworms or Colorado Potato Beetle, or a fungal pest.

The present invention also includes DNA which encodes the present inventive RNA, as well as a composition of matter, a recombinant construct or a transgenic non-human organism, tissue or cell which comprises the said RNA or DNA.

The recombinant construct according to the invention is preferably a DNA expression cassette and will most probably contain—alongside the nucleic acid encoding the RNA molecule of the invention—a promoter, terminator, selectable marker gene and other regulatory elements. The promoter might be inducible (for example one in which expression commences on application of a particular factor), or constitutive. The selectable marker gene might be an antibiotic resistance gene, including those for prokaryotes or eukaryotes. Prokaryotic antibiotic resistance markers include ampicillin, kanamycin, metronidazole, and tetracycline resistance. Eukaryotic resistance markers include neomycin, puromycin and hygromycin resistance. The selectable marker gene may encode a visual marker, such as a fluorescent protein (for example green fluorescent protein), or luciferase. The selectable marker gene may be a biochemical marker, or a morphological marker, or any another suitable marker enabling selection of an organism expressing the construct of this invention, such as a DNA sequence encoding a PAT or EPSPS enzyme which provides for herbicide based selection.

The DNA expression cassette described herein may be introduced into a host cell to produce a transgenic organism. In some embodiments the host cell is a eukaryotic cell. Suitable eukaryotic cells include, but are not limited to, fungal cells, non-human animal cells, algae and plant cells.

In some embodiments the host cell is a prokaryotic cell, such as a bacterial cell. If used as a biological pesticide the host cell may be inactivated by methods known to a man skilled in the art including, but not limited to, heat inactivation and/or chemical inactivation.

A further embodiment of the invention relates to the method of producing the RNA molecule of the present invention, comprising transcribing the DNA of the expression cassette of the present invention in a host cell or providing the DNA expression cassette in a cell free system and providing conditions that result in the transcription of the DNA to produce the RNA of this present invention.

Additionally, the RNA molecule might be isolated and/or purified from a host cell, or a cell free mix, by methods known to the man skilled in the art.

The invention still further includes a method of controlling a pest organism by post transcriptionally silencing an essential gene in the organism, comprising introducing into the said organism or the cells thereof an RNA, a DNA, or a composition or construct/cassette according to the present invention.

The invention still further provides use of an RNA according to the present invention, DNA according to the present invention, or a composition or construct according to the present invention to control the growth or proliferation of a pest organism, or the cells or tissues thereof.

The pesticidal composition of the present invention may comprise at least one RNA molecule of this present invention and/or the DNA encoding it and/or the expression construct of the present invention and/or a cell (active or inactivated) expressing the RNA molecule of the present invention. The pesticidal composition may further comprise an agronomically acceptable excipient and/or diluent and, optionally, at least one other known pesticidaly active ingredient, such as an insecticidal protein (for example a Bt crystal protein) or a small molecule (such as a pyrethroid or a neonicotinoid).

The pesticidal composition may be in any form suitable for application to a pest organism. The composition may be in solid form (powder, pellet, or a bait), liquid form, or gel form.

A further aspect of the invention relates to controlling a plant pest organism by applying to a plant and/or to the seed of a plant and/or locus of a plant or seed an RNA molecule of the present invention and/or a DNA encoding the RNA of the present invention and/or an expression construct comprising a nucleic acid molecule encoding the RNA molecule of the present invention and/or a composition of the invention.

In a further aspect the invention relates to a transgenic plant containing the nucleic acid molecule (RNA or DNA) of the present invention. The nucleic acid molecule may be introduced by routine genetic engineering techniques. Therefore, a further aspect of the invention provides a method of generating a transgenic plant containing the nucleic acid molecule of this present invention comprising: transforming a plant cell with a DNA construct encoding the RNA of this invention, regenerating said transformed plant cell into a viable plant and subsequent propagation of said plant.

Suitable methods of transformation include, but are not limited to, microinjection, agrobacterium-mediated transformation, micro-projectile bombardment, electroporation, lipofection, polymer based transfection and viral transduction.

The present invention will be further apparent from the following non-limiting exemplification in which FIGS. 1 to 6 show the percentage mortality at day 7 after treatment, induced by different doses of in vitro synthesized hairpin samples for target DVs006.5 and in which FIGS. 7 to 9 show the percentage mortality at day 7 after treatment, induced by different doses of in vitro synthesized hairpin samples for target DVs004.4, and FIG. 10, which shows the sequences of the DNAs which were tested.

Materials and Methods:

Generation of Test Samples:

Generation of Template

The DNA template for the dsRNA synthesis was designed in silico using Informax Vector NTI® 11 package. The sequence of interest was ordered at a third party and cloned into a backbone vector containing a T7 promoter.

Different types of modifications can be introduced into the DNA; the currently used approaches are listed in Table 1. With modification 1 every Adenine is changed into a Thymine and vice versa.

TABLE 1
Overview and description of the different
modifications in the DNA template.

| Modification | Description |
|---|---|
| Mod 1 (A/T) | Every adenine is changed into a thymine |
| Mod 2 (1/10) | Every 10^{th} nucleotide is |
| | changed according to table 2 |
| Mod 3-5 (1/21) | Every 21st nucleotide is |
| | changed according to table |
| | 2, counting starts with 1 bp |
| | difference for mod4 and |
| | mod5 |
| Mod 6 (1/5) | Every 5^{th} nucleotide is changed |
| | according to table 2 |
| Mod 7 (8-9-10th) | Every 8, 9 and 10^{th} nucleotide is |
| | changed according to table 2 |
| GFP instead of target | The target sequence is completely |
| | replaced by GFP |
| Unmodified | Original target sequence |

With modification 2, every 10^{th} nucleotide in the DNA is modified. If the 10^{th} nucleotide is a cytosine, it is changed into a thymine (and vice versa). If it is an Adenine, it is changed into a guanine (and vice versa). From modification 3 onwards, the same strategy was applied. The modifications are summarized in Table 2.

TABLE 2
Summary of how the modifications are established.
Modification

C > T
A > G
T > C
G > A

Modifications were introduced in the sense strand and/or the antisense strand of the hairpin (corresponding to the first and second or second and first sequences of the RNA, as the case may be—depending on whether the transcription in the 5′ to 3′ direction corresponds to the expression of the second sequence before or after the spacer sequence.

The template for in vitro transcription was generated by linearizing the plasmid behind the hairpin cassette using an appropriate restriction enzyme.

In Vitro dsRNA Transcription

dsRNA was generated using the Ribomax T7 kit (Promega cat n#1230) according to the manufacturer's protocol.

In short the in vitro transcription reaction was set up by mixing the template with the transcription buffer and the T7 polymerase from the kit. This mix is incubated over night at 37° C. After incubation, the dsRNA was heated to 70° C. and cooled down at room temperature to allow annealing of the two strands. After that, a DNase treatment was performed to remove the template. The RNase treatment is omitted. A final purification step was performed using alcohol precipitation.

The material is finally dissolved in MilliQ water. Concentration is determined using the Trinean Dropsense 96.

Corn Rootworm Diet Based Assay

Diet plates was prepared by adding 500 μl artificial diet per well in 48 well-plates. The dsRNA sample was added in a minimum volume of 20 μl on the surface of the diet in order to cover the whole surface. The plates were then left to dry in the laminar flow hood.

A single neonate larva was placed on the surface of every well using a paintbrush. After that the plates were sealed (Greiner cat. N#676070). Air holes were generated by punching the seal. The plates are incubated in dark containers, at 26° C., under 65% humidity.

The larvae were checked daily for lethality and or phenotypes. Data are normalized against the background mortality that is observed on day 1 after set-up.

Results

Assessment of the bioactivity of the modified hairpin samples was performed in the Western Corn Rootworm Diet based assay. The samples were tested at different doses per well (ranging from 1 μg down to 0.001 μg per well). 24 to 40 larvae were assessed per treatment.

Modified Hairpins for Target Dvs006.5

The unmodified hairpin for Corn rootworm target Dvs006.5 (designated as “original hp” in the figures below) gave over 90% mortality at day 7 with doses down to 0.01 μg per well. When every Adenine was replaced by a Thymine (and vice versa) in the sense strand of the hairpin, a similar bio activity was observed (see FIG. 1).

When every tenth nucleotide in the sense strand was modified (see Table 1), over 90% mortality was observed at day 7 with doses down to 0.1 μg per well (see FIG. 2).

When every tenth nucleotide was modified in the antisense strand or in both strands, a significant reduction or even no bio activity was observed (FIG. 3).

The hairpin sample with every 21^{st} nucleotide modified in the sense strand, only gave 90% mortality at day 7 at the highest dose of 1 μg per well (see FIG. 4)

When modifications were introduced at a higher frequency then 1/10 nucleotides, for example every 5^{th} (FIG. 5) or every 8^{th}, 9^{th} and 10^{th} nucleotide (FIG. 6), a significant reduction in bioactivity was observed.

Any modification in the antisense strand, i.e. the sequence which hybridizes with the mRNA of the target gene, led to loss of activity of the dsRNA molecule (FIGS. 3, 5 and 6). This is in line with the notion that RNAi is sequence dependent and very specific.

Modified Hairpins for Target Dvs004.4

The unmodified hairpin for Corn rootworm target Dvs004.4 (designated as “original hp” in the figures below) gave around 80% mortality at day 7 with doses down to 0.1 μg per well.

A similar bioactivity was observed when every 21^{st} nucleotide of the sense strand was modified (FIG. 8). The hairpin sample with a modification in every tenth nucleotide of the sense strand only showed more than 75% mortality on day 7 at the highest dose of 1 μg (FIG. 7).

Modification of every 8^{th}, 9^{th} and 10^{th} nucleotide of the sense strand (mod7) resulted in a severe reduction of the bio activity and none of the treatments induced more than 75% mortality at day 7.

### FIGURE LEGENDS

FIG. 1: Percentage mortality at day 7 after treatment induced by different doses of in vitro synthesized hairpin samples for target Dvs006.5. Data for hairpin dsRNA samples with every Adenine was replaced by a Thymine (and vice versa) in the sense strand of the hairpin (black bars). Negative controls (striped bars): diet alone and non-target dsRNA (NC). Positive control: the unmodified hairpin (original hairpin, white bars).

FIG. 2: Percentage mortality at day 7 after treatment induced by different doses of in vitro synthesized hairpin samples for target Dvs006.5. Data for hairpin dsRNA samples with modifications in every tenth nucleotide of the sense (black bars), antisense (grey bars) or both strands (dotted bars) are represented. Negative controls (striped bars): diet alone and non-target dsRNA (NC). Positive control: the unmodified hairpin (original hairpin, white bars).

FIG. 3: Percentage mortality at day 7 after treatment induced by different doses of in vitro synthesized hairpin samples for target Dvs006.5. Data for hairpin dsRNA samples with modifications in every tenth nucleotide of the sense (black bars), antisense (grey bars) or both strands (dotted bars) are represented. Negative controls (striped bars): diet alone and non-target dsRNA (NC). Positive control: the unmodified hairpin (original hairpin, white bars).

FIG. 4: Percentage mortality at day 7 after treatment induced by different doses of in vitro synthesized hairpin samples for target Dvs006.5. Data for hairpin dsRNA samples with every 21st nucleotide modified in the sense strand of the hairpin (black bars). Negative controls (striped bars): diet alone and non-target dsRNA (NC). Positive control: the unmodified hairpin (original hairpin, white bars).

FIG. 5: Percentage mortality at day 7 after treatment induced by different doses of in vitro synthesized hairpin samples for target Dvs006.5. Data for hairpin dsRNA samples with modifications in every fifth nucleotide of the sense (black bars), antisense (grey bars) or both strands (dotted bars) are represented. Negative controls (striped bars): diet alone and non-target dsRNA (NC). Positive control: the unmodified hairpin (original hairpin, white bars).

FIG. 6: Percentage mortality at day 7 after treatment induced by different doses of in vitro synthesized hairpin samples for target Dvs006.5. Data for hairpin dsRNA samples with modifications in every 8th, 9th and 10th nucleotide (Mod7) of the sense (black bars), antisense (grey bars) or both strands (dotted bars) are represented. Negative controls (striped bars): diet alone and non-target dsRNA (NC). Positive control: the unmodified hairpin (original hairpin, white bars).

FIG. 7: Percentage mortality at day 7 after treatment induced by different doses of in vitro synthesized hairpin samples for target Dvs004.4. Data for hairpin dsRNA samples with modifications in every tenth nucleotide of the sense (black bars), antisense (grey bars) or both strands (dotted bars) are represented. Negative controls (striped bars): diet alone and non-target dsRNA (NC). Positive control: the unmodified hairpin (original hairpin, white bars).

FIG. 8: Percentage mortality at day 7 after treatment induced by different doses of in vitro synthesized hairpin samples for target Dvs004.4. Data for hairpin dsRNA samples with every 21st nucleotide modified in the sense strand of the hairpin (black bars). Negative controls (striped bars): diet alone and non-target dsRNA (NC). Positive control: the unmodified hairpin (original hairpin, white bars).

FIG. 9: Percentage mortality at day 7 after treatment induced by different doses of in vitro synthesized hairpin samples for target Dvs004.4. Data for hairpin dsRNA samples with modifications in every 8th, 9th and 10th nucleotide (Mod7) of the sense strand are represented (black bars). Negative controls (striped bars): diet alone and non-target dsRNA (NC). Positive control: the unmodified hairpin (original hairpin, white bars).

FIG. 10: The hairpin sequences tested.

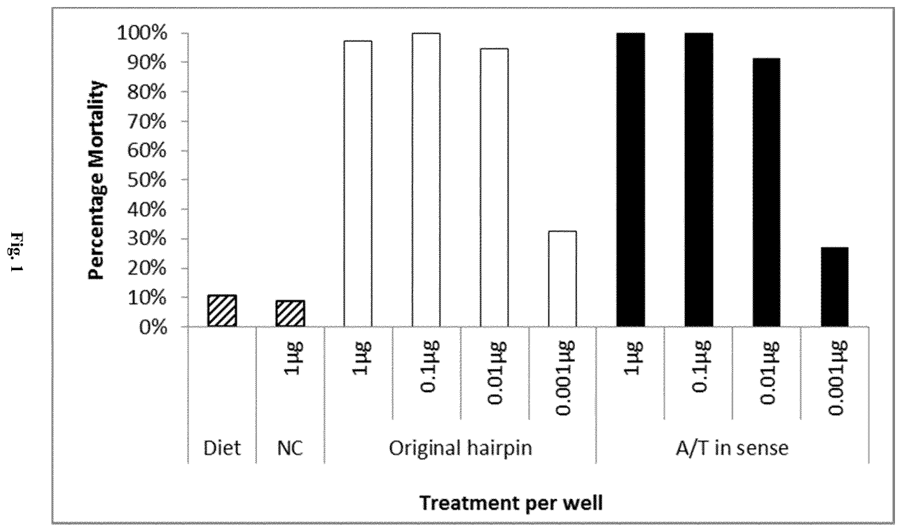

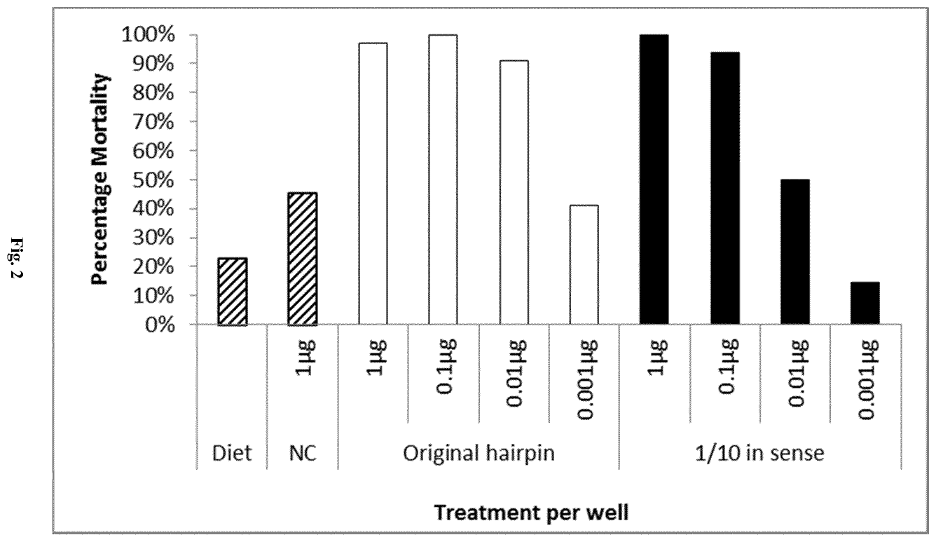

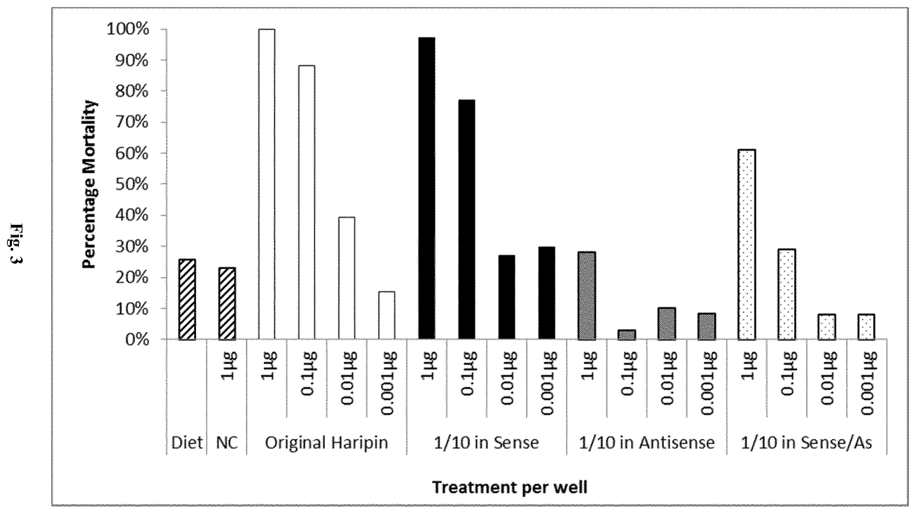

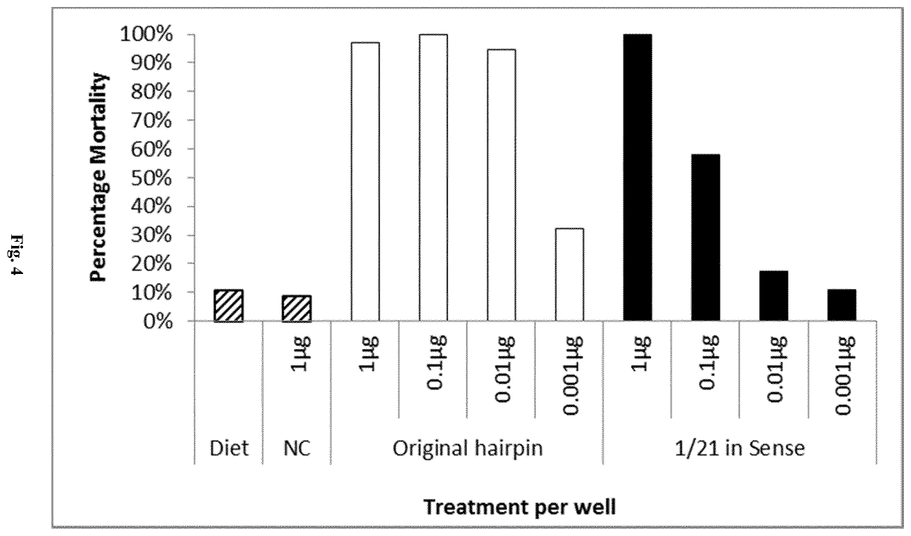

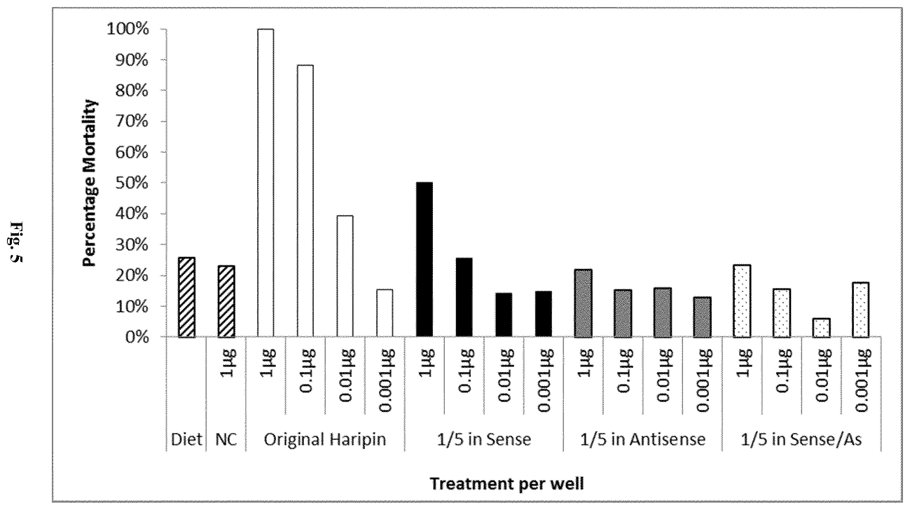

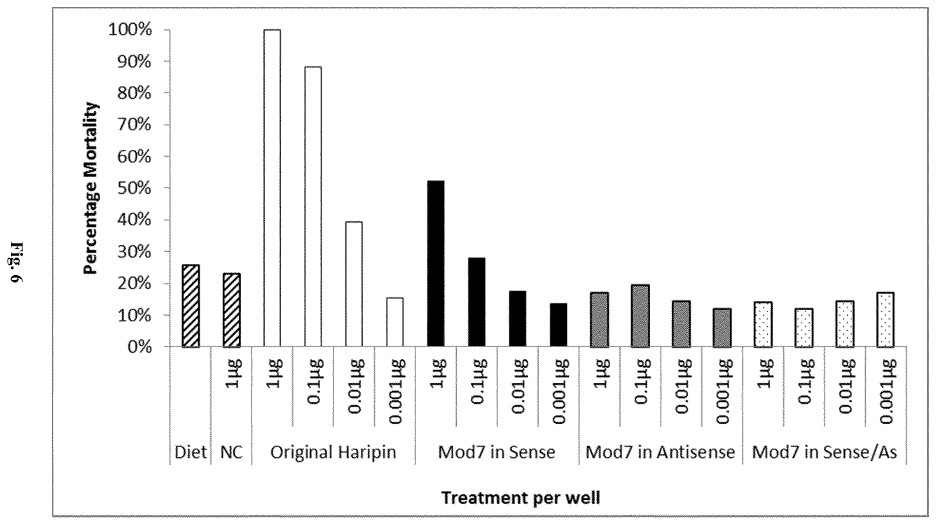

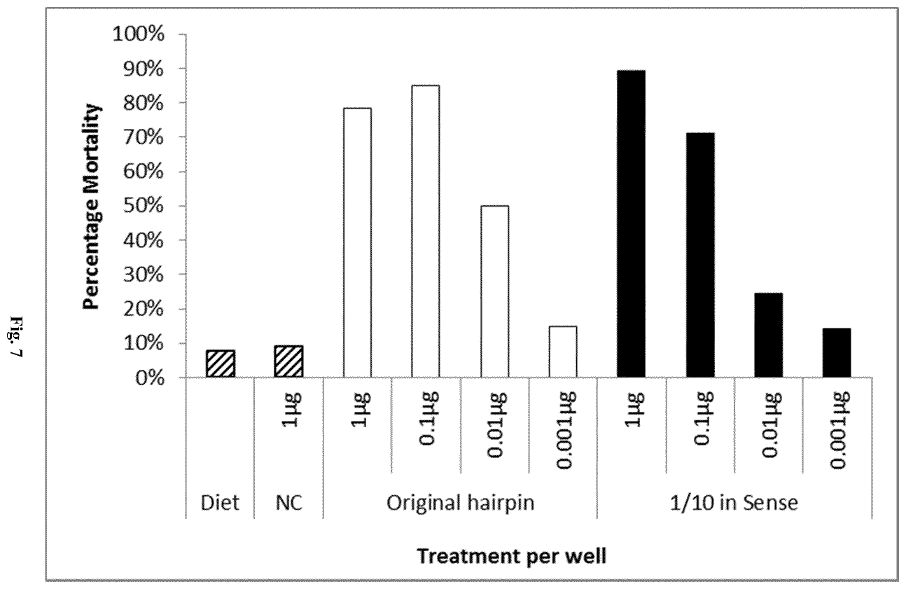

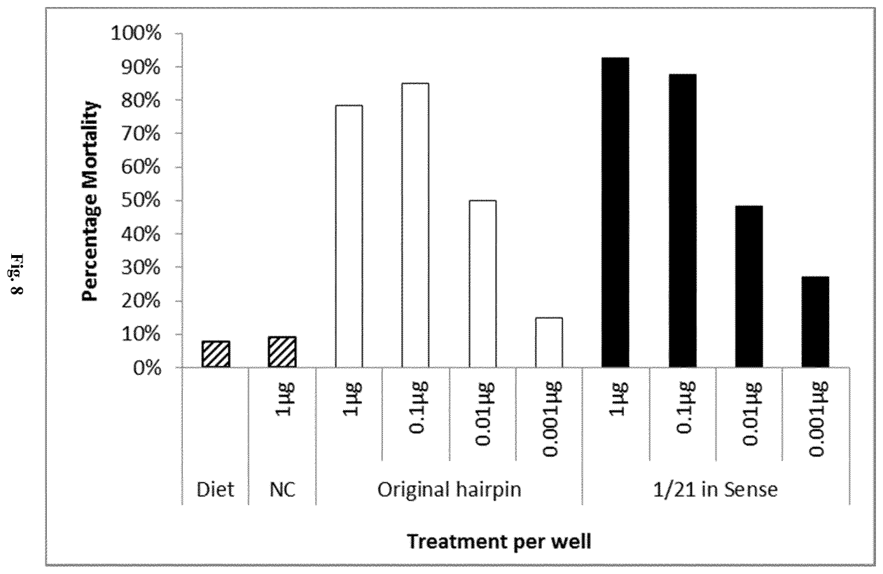

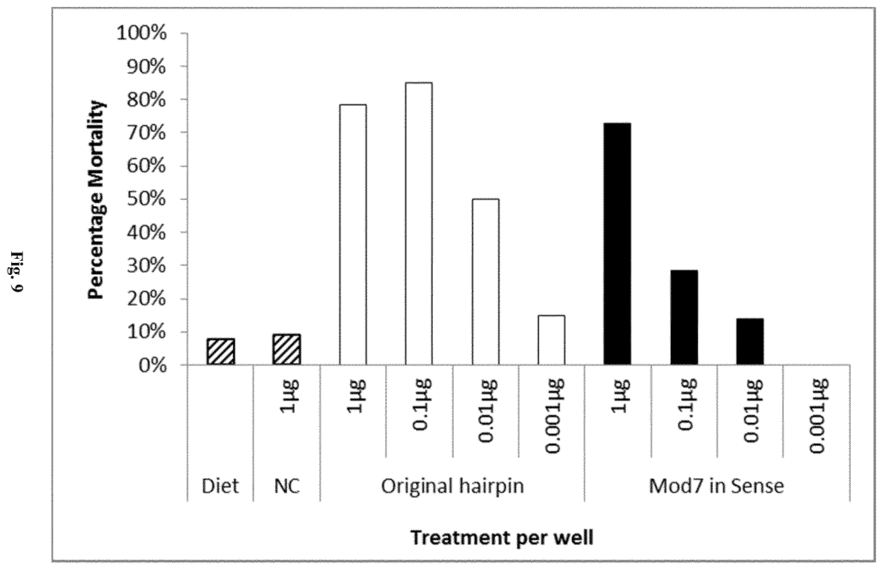

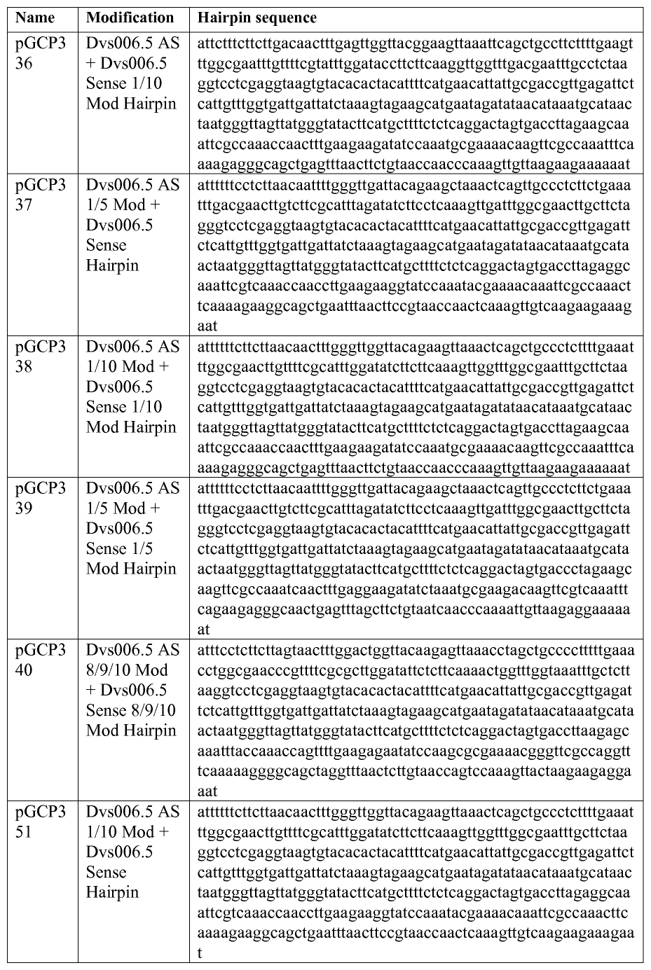

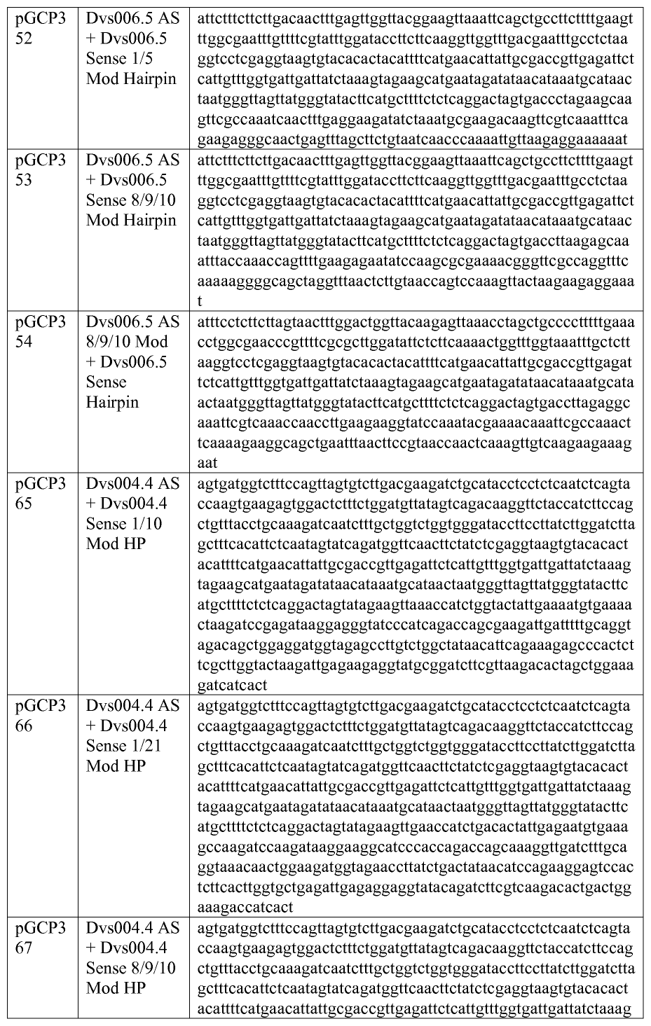

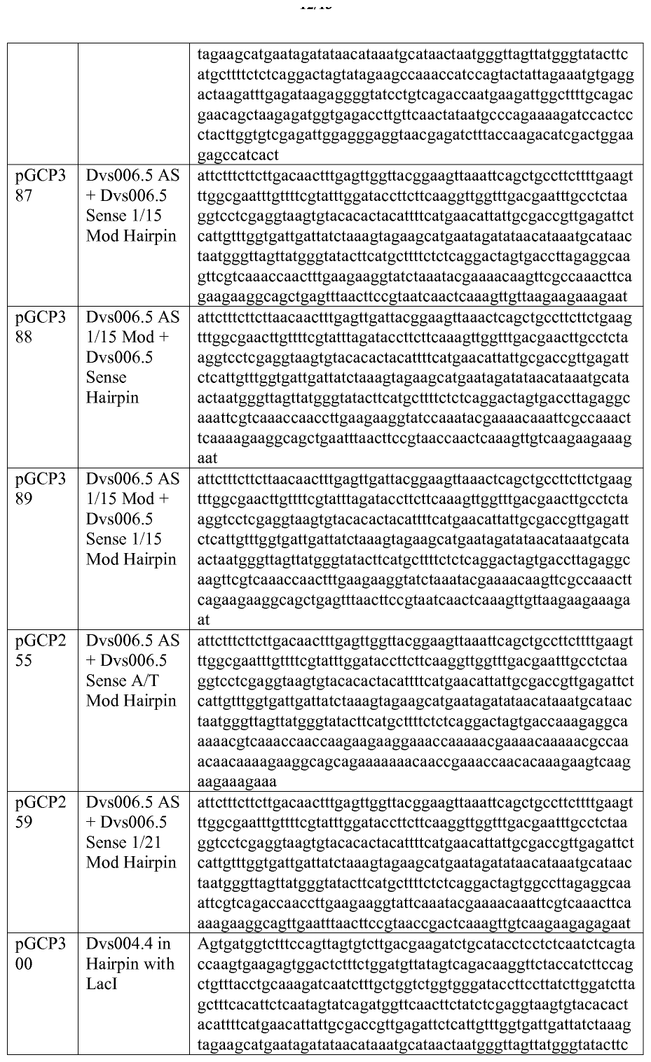

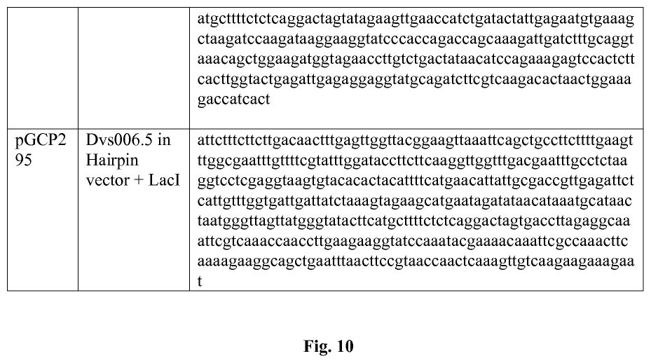

1. A hairpin RNA (hpRNA) comprising in the 5′ to 3′ direction a first sequence and a second sequence that are substantially complementary and form a duplex, the first sequence being spaced apart from the second sequence by a spacer sequence, wherein (a) the second sequence is able to hybridize with a mRNA of a target gene in an organism, and is fully complementary to the mRNA along a region at which it hybridizes, and wherein the first sequence comprises nucleotide bases which do not base pair with the corresponding bases in the second sequence of the duplex; or (b) the first sequence is able to hybridize with a mRNA of a target gene in an organism and is fully complementary to the mRNA along a region at which it hybridizes, and wherein the second sequence comprises nucleotide bases which do not base pair with the corresponding bases in the first sequence of the duplex, wherein the duplex does not comprise regions of 3 or more consecutive mismatched bases.

2. (canceled)

3. A hpRNA according claim 1, wherein the mismatching bases are substantially equally spaced along the length of the duplex.

4. A hpRNA according to claim 1, wherein the mismatching bases are spaced along the length of the duplex about every 5th position, or at about every 10th position or at about every 20th position.

5. A hpRNA according to claim 1, wherein the duplex comprises about 20 percent of nucleotides which do not base pair.

6. A hpRNA according to claim 1, wherein the duplex comprises about 10 percent of nucleotides which do not base pair.

7. A hpRNA according to claim 1, wherein the nucleotides which do not base pair are A in the first sequence which corresponds with C in the second sequence, U in the first sequence which corresponds with G in the second sequence, C in the first sequence which corresponds with A in the second sequence.

8. A hpRNA according to claim 1, wherein the spacer sequence has a length of about 15 to about 350 nucleotides, or a length of about 20 to about 100 nucleotides or a length of about 20 to about 50 nucleotides.

9. A hpRNA according to claim 1, wherein the length of the duplex in the hairpin structure is about 2000 nucleotides, or about 1500 nucleotides or about 100 to 500 nucleotides.

10. A hpRNA according to claim 1, wherein the length of the sequence which hybridizes with the mRNA is about 20 to about 100 nucleotides, or about 20 to about 80 nucleotides or about 20 to about 30 nucleotides.

11. A hpRNA according to claim 1, wherein the target gene is an essential gene of a plant or a plant pest organism.

12. A DNA molecule which encodes the hpRNA of claim 1.

13. A recombinant construct which comprises the DNA molecule of claim 12.

14. A method of controlling a pest organism by post transcriptionally silencing an essential gene in the organism, comprising introducing into the organism the hpRNA according to claim 1.

15. (canceled)

16. A transgenic non-human organism, tissue or cell comprising the DNA molecule of claim 12.

17. A transgenic non-human organism, tissue or cell comprising the hpRNA of claim 1.
